(19) United States
(12) Patent Application Publication
(10) Pub. No.: US 2024/0049949 A1
(43) Pub. Date: Feb. 15, 2024

(54) DIRECT PERORAL CHOLANGIOSCOPE SYSTEM WITH GUIDE SHEATH

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MA (US); Thomas J. Holman, Princeton, MN (US); Anthony R. Pirozzi, Raleigh, NC (US)

(21) Appl. No.: 18/257,792

(22) PCT Filed: Dec. 14, 2021
(86) PCT No.: PCT/US2021/063303

§ 371 Date: Jun. 15, 2023

Related U.S. Application Data
(60) Provisional application No. 63/127,390, filed on Dec. 18, 2020.

(51) Int. Cl. A61B 1/00; A61B 1/018; A61B 1/05; A61B 10/06

(52) U.S. Cl. CPC A61B 1/00135; A61B 1/018; A61B 1/05; A61B 10/06

(57) ABSTRACT

A direct peroral cholangioscope system comprises a guide sheath sized to pass through an esophagus of a patient comprising a proximal end portion, a distal end portion, and a lumen extending between the proximal end portion and the distal end portion, an endoscope comprising an elongate body extending from a proximal end to a distal end, an imaging device coupled to the elongate body proximate the distal end, and a passage extending at least partially through the elongate body and exiting at the distal end, the elongate body sized to pass through the lumen, and a tissue retrieval device configured to pass through the passage and retrieve tissue from within the patient. The guide sheath can be used to steer the endoscope through the pyloric sphincter and the sphincter of Oddi to allow the endoscope to be articulated in the common bile duct using native steering capabilities.

### CROSS-REFERENCE TO RELATED APPLICATION

This application claims the benefit of priority to U.S. Provisional Patent Application Ser. No. 63/127,390, filed Dec. 18, 2020, the contents of which are incorporated herein by reference in their entirety.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices, such as endoscopes, that can be inserted into anatomy of a patient, such as with the aid of another device, to facilitate performance of a biological matter removal process, such as by cutting sample tissue with a forceps for later analysis.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

One example of an endoscopic procedure is called an Endoscopic Retrograde Cholangio-Pancreatography, hereinafter “ERCP” procedures. In an ERCP procedure, an auxiliary scope (also referred to as daughter scope, or cholangioscope) can be attached and advanced through the working channel of a “main scope” (also referred to as mother scope or duodenoscope). Furthermore, a tissue retrieval device used to remove the sample matter is inserted through the auxiliary scope. As such, the duodenoscope, auxiliary scope and tissue retrieval device become progressively smaller, due to being sequentially inserted in progressively smaller lumens, and more difficult to maneuver and perform interventions and treatments.

### SUMMARY

The present inventors have recognized that problems to be solved with conventional medical devices, and in particular endoscopes and duodenoscopes used to retrieve sample biological matter, include, among other things, 1) the difficulty in navigating endoscopes, and instruments inserted therein, to locations within anatomical regions deep within a patient, 2) the disadvantage associated with operating three instruments (e.g., duodenoscope, cholangioscope, tissue removal device) such as the need for multiple skilled instrument operators, 3) the increased time and associated cost of having to repeatedly remove and reinsert medical devices to obtain a sufficient quantity of sample material, and 4), the difficulty of incorporating features (e.g., steerability and tissue collection features) into small-diameter devices.

The present inventors have recognized that such problems can be particularly present in duodenoscopy procedures such as the aforementioned ERCP procedures. Recent attempts have been made to address the deficiencies of ERCP procedures. Recent developments have involved the use of endoscopes in direct peroral cholangioscopy procedures where an endoscope is advanced directly into the mouth of a patient to reach the common bile duct. See for example the discussion in Direct Peroral Cholangioscopy by Mansour A. Parsi, MD, in World Journal of Gastrointestinal Endoscopy published online Jan. 16, 2014. However, such procedures are difficult to perform due looping of the endoscope produced by navigating the endoscope through the pyloric sphincter of the stomach and the sphincter of the common bile duct (Sphincter of Oddi). This looping of the endoscope can result in inoperability of the endoscope due to binding, e.g., the endoscope is too tightly curved to allow for additional articulation.

The present disclosure can help provide solutions to these and other problems by providing systems, devices and methods relating to direct peroral cholangioscopy procedures, such as those including a guide sheath that can be placed over an endoscope to provide 1) direct secondary steerability of the endoscope in addition to the primary native steering capabilities of the endoscope and 2) a rigid structure against which the endoscope can be pushed using the primary native steering capabilities to provide for articulation of the endoscope. As such, when the endoscope is positioned in the common bile duct, the native steerability features of the endoscope remain operable to position a therapeutic device at target tissue sites.

As such, the present disclosure can help solve the problems referenced above and other problems by 1) increasing the ease of use of direct per oral cholangioscopy systems and ERCP systems (e.g., fewer operators, less required skill to navigate through the Sphincter of Oddi/ampulla of Vater), and 2) increasing treatment device size (e.g., increasing the volume of sample material collected with each insertion, reducing the number of times a tissue retrieval device needs to be inserted and reinserted into the anatomy), among other things, as is described herein.

In an example, a direct peroral cholangioscope system can comprise a guide sheath, an endoscope and a tissue retrieval device. The guide sheath can comprise a proximal end portion, a distal end portion, and a lumen extending between the proximal end portion and the distal end portion, wherein the guide sheath is sized to pass through an esophagus of a patient. The endoscope can comprise an elongate body extending from a proximal end to a distal end, an imaging device coupled to the elongate body proximate the distal end, and a passage extending at least partially through the elongate body and exiting at the distal end, wherein the elongate body is sized to pass through the lumen. The tissue retrieval device can be configured to pass through the passage and retrieve tissue from within the patient.

In another example, a method of performing a direct peroral cholangioscopy procedure can comprise inserting an endoscope into a guide sheath, inserting the guide sheath and endoscope into the stomach of a patient, inserting the guide sheath and endoscope into the duodenum of the patient, turning a distal end of the endoscope with the guide sheath to position the endoscope proximate the sphincter of Oddi, inserting a tissue retrieval device into the endoscope, positioning the tissue retrieval device in the common bile duct of the patient, and collecting biological matter with the tissue retrieval device.

### BRIEF DESCRIPTION OF DRAWINGS

FIG. 1A is a schematic diagram of a direct peroral cholangioscope system comprising a guide sheath, a cholangioscope and a tissue retrieval device in a nested configuration.

FIG. 1B is a schematic diagram of the direct peroral cholangioscope system of FIG. 1A in an exploded state showing internal lumens through the guide sheath and the cholangioscope.

FIG. 2 is a schematic diagram of the distal end portions of the guide sheath, the cholangioscope, and the tissue retrieval device of FIGS. 1A and 1B.

FIG. 3 is a schematic diagram of the cholangioscope of FIGS. 1A and 1B showing a schematic representation of an imaging and control system comprising a control unit connected to the cholangioscope.

FIG. 4 is schematic diagram of the imaging and control system of FIG. 3 connected to the cholangioscope.

FIG. 5A is an end view of a camera module including optical and functional components suitable for use with the cholangioscope of FIGS. 1A-4.

FIG. 5B is a cross-sectional view taken along the plane 5B-5B of FIG. 5A showing components of the camera module.

FIG. 6 is a schematic diagram illustrating the guide sheath and the cholangioscope of FIGS. 1A and 1B inserted perorally into a patient to reach the duodenum.

FIG. 7 is a schematic diagram illustrating the guide sheath and the cholangioscope of FIG. 6 positioned in the duodenum and the tissue retrieval device positioned in the common bile duct.

FIG. 8 is a block diagram illustrating methods of retrieving tissue samples from within a common bile duct of a patient using a guide sheath for a direct peroral cholangioscope according to examples of the present disclosure.

### DETAILED DESCRIPTION

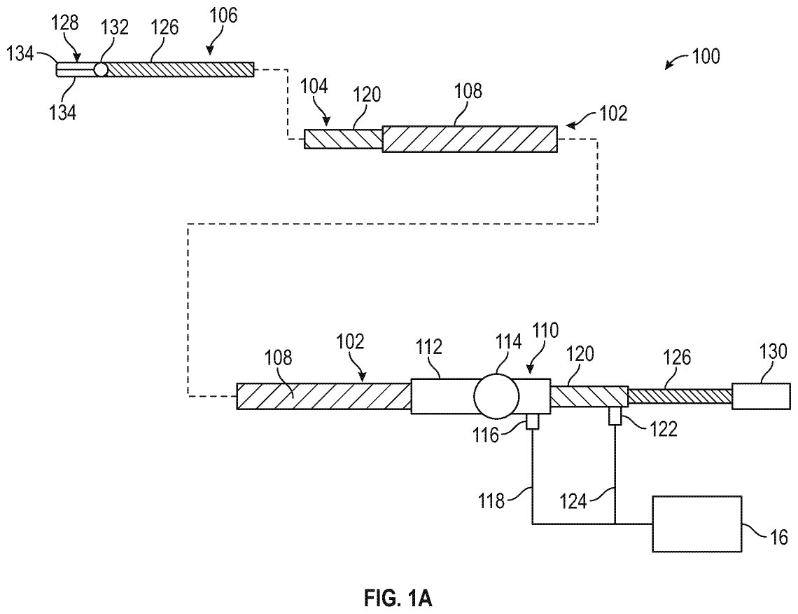

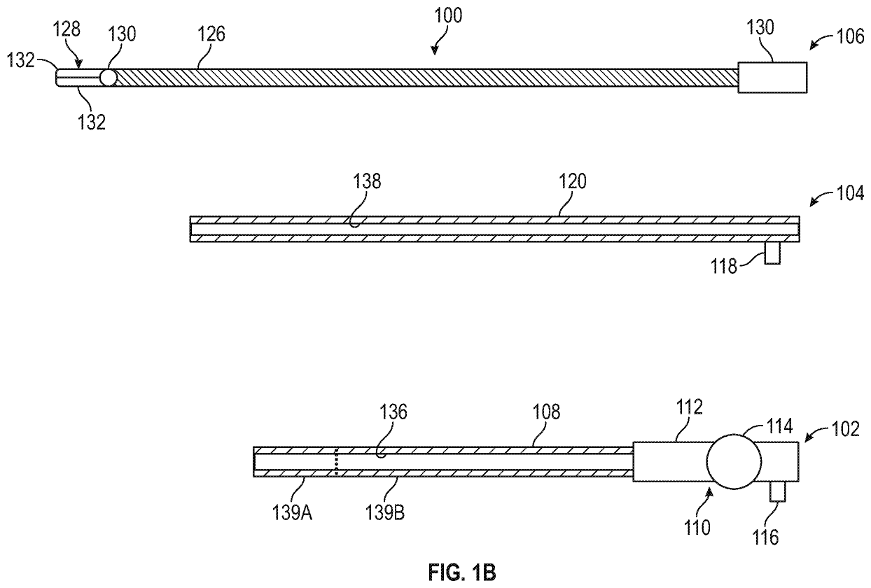

FIG. 1A is a schematic diagram of direct peroral cholangioscopy system 100 wherein guide sheath 103, cholangioscope 104 and tissue retrieval device 106 are in a nested configuration. FIG. 1B is a schematic diagram of direct peroral cholangioscopy system 100 wherein guide sheath 103, cholangioscope 104 and tissue retrieval device 106 are in an exploded configuration. FIGS. 1A and 1B are discussed concurrently. FIGS. 1A and 1B are not necessarily drawn to scale and may be exaggerated in certain aspects for illustrative purposes.

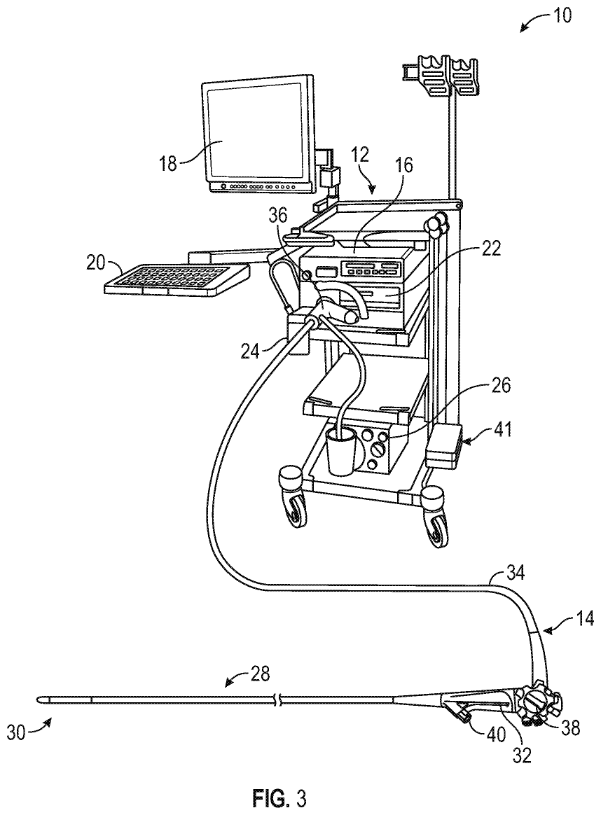

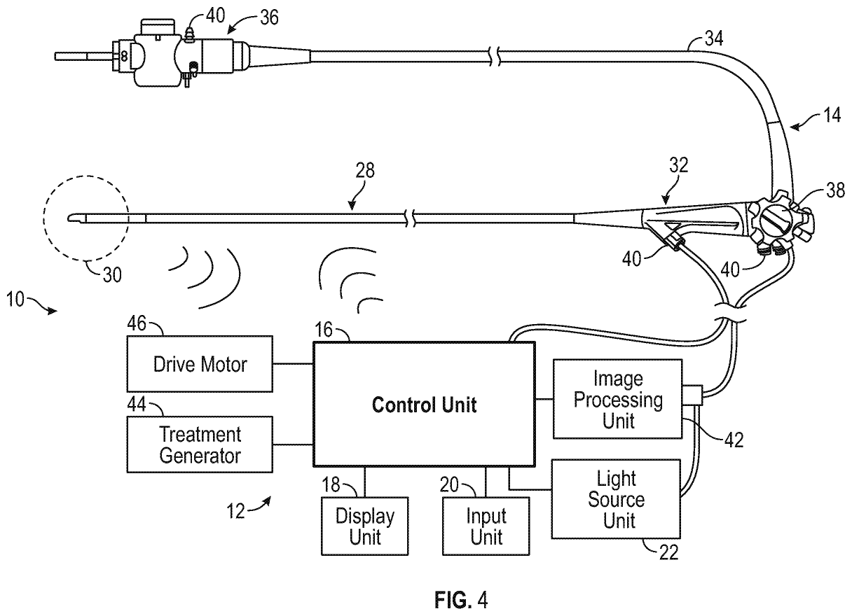

System 100 can comprise guide sheath 102, cholangioscope 104 and tissue retrieval device 106. Sheath 102 can comprise shaft 108 and control device 110, which can include grip 112, control knob 114 and coupler 116 that can connect to control unit 16 (FIG. 4) via cable 118. Cholangioscope 104, which is described in greater detail with reference to FIGS. 3-5B, can comprise elongate body 120 and coupler 122 that can connect to control unit 16 via cable 124. Tissue retrieval device 106 can comprise shaft 126, tissue separator 128 and control device 130. Tissue separator 128 can comprise hinge 132 and separators 134.

FIG. 1A shows cholangioscope 104 nested inside of sheath 102, and tissue retrieval device 106 nested inside cholangioscope 104. As such, as can be seen in FIG. 1B, sheath 102 can comprise lumen 136 and cholangioscope 104 can comprise lumen 138.

As is discussed in greater detail herein, direct peroral cholangioscopy system 100 can be configured to provide simplified navigation to a duodenum and common bile duct, and allow for large sample sizing, thereby reducing procedure complexity and the number of times that an instrument is inserted into the duodenum and common bile duct to retrieve an adequate amount of tissue upon which testing can be performed.

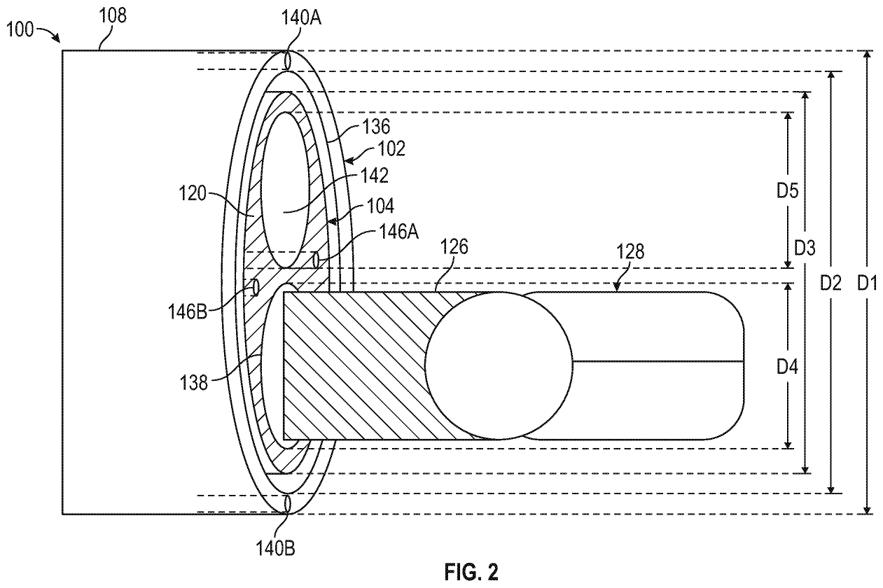

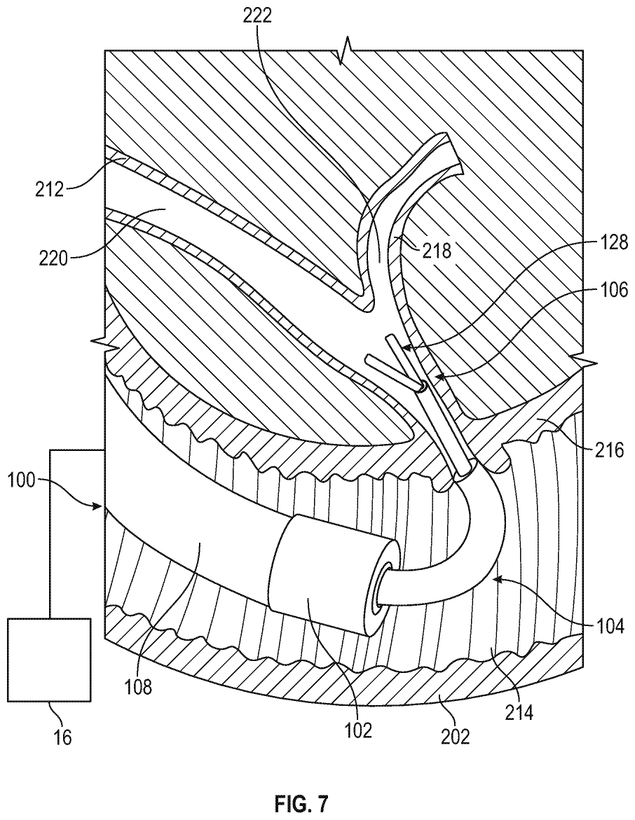

Shaft 108 of guide sheath 102 can include pull wires (140A, 140B of FIG. 2) that can be used to steer guide sheath 102. Control device 110 can be used to operate guide sheath 102, including the pull wires. For example, grip 112 can be grasped by an operator and control knob 114 can be rotated to pull on one or both of the pull wires to apply directionality to the shape of shaft 108. Guide sheath 102 can thus be used to influence the shape of cholangioscope 104 that can be positioned inside of shaft 108. Thus, shaft 108 can be made of suitable materials compliant enough to be directed by pull wires, but rigid enough to allow cholangioscope 104 to push off of shaft 108. In examples, shaft 108 can be configured in sections having different rigidities to, for example, concentrate the ability of shaft 108 to be steered in a particular section of guide sheath 102. For example, distal end portion 139A of shaft 108 (e.g., approximately the most-distal 10 ten to twenty percent) can be configured to be more flexible than the remaining proximal portion 139B so that the pull wires can pull the distal end of shaft 108 at a sharp angle (e.g., approximately thirty to ninety degrees), which can be useful in guiding cholangioscope 104 into common bile duct 212 (FIG. 7) from duodenum 202 (FIG. 7). Sectioning of the flexibility of shaft 108 can allow more rigid portions proximal of the distal flexible portion to provide stiffening to cholangioscope to allow cholangioscope to use native steering capabilities more effectively (e.g., without binding as described above). For example, proximal portion 139B can have a rigidity that is more rigid than cholangioscope 104 so that when cholangioscope 104 is steered, e.g., by tensioning of one or more pull wires 146A and 146B, cholangioscope 104 can be pushed into the desired direction by the pulling of pull wires 146A and 146B.

Cholangioscope 104 can be configured as a fully functional endoscope including steerability, guidance capability, imaging capability, fluid dispensing and retrieving capabilities, and functional (e.g., therapeutic and diagnostic) capabilities, as well as a passageway for other instruments. Functionality of cholangioscope 104 is described in detail with reference to endoscope 14 of FIGS. 3 and 4 below and, as such, is only shown schematically in FIGS. 1A and 1B.

Tissue retrieval device 106 can be configured as any suitable device configured to obtain tissue samples form within a patient. However, tissue retrieval device 106 can comprise a component or device for interacting with a patient, such as those configured to cut, slice, pull, saw, punch, twist or auger tissue, and the like. Specifically, tissue retrieval device 106 can comprise any device suitable for removing tissue from a patient, such as a blade, punch or an auger. Tissue retrieval device 106 can be configured to physically separate portions of tissue of a patient from other larger portions of tissue in the patient. In additional examples, tissue retrieval device 106 can be configured to simply collect biological matter from the patient that does not need physical separation, such as mucus or fluid. In the illustrated example, tissue retrieval device 106 can comprise forceps having separators 134 configured as sharpened or serrated jaws pivotably connected at hinge 132. Tissue retrieval device 106 can, however, be configured as a variety of devices capable of collecting biological matter, such as a punch, an auger, a blade, a saw and the like, as mentioned. The term “tissue retrieval device” is used throughout the present disclosure, however tissue retrieval device 106 can alternatively or additionally comprise a biological matter collection device, a biological matter retrieval device, a tissue collection device and tissue retrieval device.

Tissue retrieval device 106 can be configured to hold a volume of collected biological matter, e.g., tissue, such as between separators 134. As such, tissue retrieval device 106 can be configured to be withdrawn from cholangioscope 104 to obtain the collected biological matter, such as for diagnostic analysis. In other examples, tissue retrieval device 106 can include a lumen through which biological matter can be withdrawn without having to withdraw tissue retrieval device 106 from cholangioscope 104.

Guide sheath 102 can be configured as a simple tubular body with limited navigation capabilities that can be used to enhance native navigation capabilities of a cholangioscope, without increasing the size and complexity of the system components and the procedure, thus allowing the native navigation and imaging capabilities of cholangioscope 104 to be used throughout the procedure. As such, a mother duodenoscope that requires a small-sized cholangioscope, associated electronics and associated skilled personnel can be eliminated. Thus, without the bulk of a full-functionality duodenoscope, the diameter of elongate body 120 of cholangioscope 104 can be increased, thereby permitting an associated increase in the size of tissue retrieval device 106. Because of the simplicity of guide sheath 102, guide sheath 102 can be configured to be disposable.

FIG. 2 is a schematic diagram of the distal end portions of guide sheath 102, cholangioscope 104, and tissue retrieval device 106 of FIGS. 1A and 1B. Guide sheath 102 can comprise shaft 108 and lumen 136. Cholangioscope 104 can comprise elongate body 120 and lumen 138. Tissue retrieval device 106 is additionally shown extending from lumen 138. Tissue retrieval device 106 can comprise shaft 126 and tissue separator 128. FIG. 2 is not necessarily drawn to scale and may be exaggerated in certain aspects for illustrative purposes.

As illustrated, guide sheath 102, cholangioscope 104 and tissue retrieval device 106 can be configured to be inserted through particular anatomy and nested within each other to maximize the size of tissue retrieval device 106.

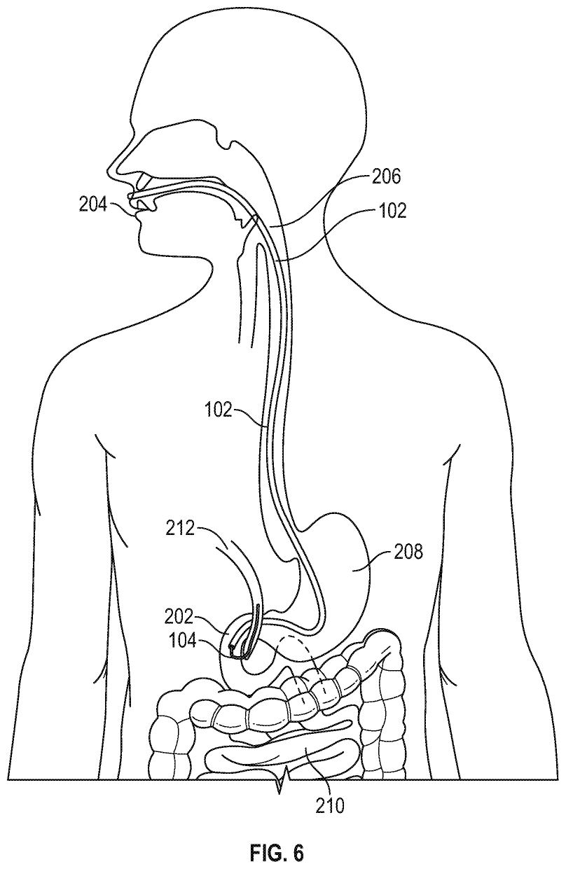

Guide sheath 108 can have outer diameter D1 configured for insertion into mouth 201 (FIG. 6) of a patient. Guide sheath 108 can be configured to be as large as anatomy of typical patients will accept, such as esophagus 206 (FIG. 6) and duodenum 202 (FIG. 6). Additionally, guide sheath 108 can be configured in different sizes to be compatible with human anatomy of different sizes. In examples, D1 can be in the range of approximately 10.0 mm to approximately 12.0 mm. In additional examples, D1 can be in the range of approximately 8.0 mm to approximately 9.0 mm.

Lumen 136 of guide sheath 108 can have diameter D2 and can be configured to be as large as feasibly possibly taking into account the desired steerability of guide sheath 108, including flexibility, durability and rigidity. Shaft 108 can have a thickness between D1 and D2 that is adequate to incorporate pull wires 140A and 140B and other associated components, such as pull rings. Control knob 114 (FIG. 1A) can be configured to apply tension to pull wires 140A and 140B to apply a bending force to shaft 108 to, for example, steer guide sheath 102 and apply an associated bending force to cholangioscope 104 when cholangioscope 104 is inserted within lumen 136. However, in other examples, guide wires can be included inside lumen 136. In examples, given the simple construction of guide sheath 108, guide sheath 108 can be configured as a disposable item intended for single use. In examples, D2 can be in the range of approximately 9.0 mm to approximately 11.00 mm. In additional examples, D2 can be in the range of approximately 8.0 mm to approximately 9.00 mm.

Cholangioscope 104 can have outer diameter D3 configured for insertion into lumen 136 of guide sheath 102. Cholangioscope 104 need not be configured to maximize size available from diameter D2, but can be configured to utilize as much of the space of lumen 136 as is necessary to provide a cholangioscope of desired capabilities. Elongate body 120 can be configured to freely slide within lumen 136 without binding. In examples, D3 can be in the range of approximately 8.0 mm to approximately 10.0 mm. In additional examples, D3 can be in the range of approximately 4.0 mm to approximately 4.5 mm.

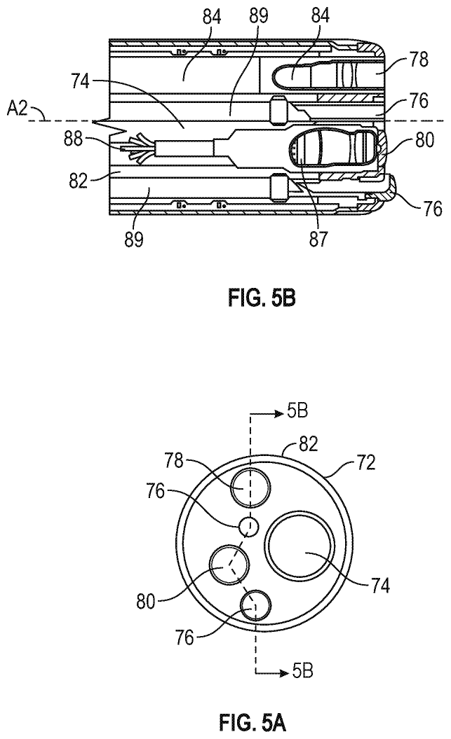

Lumen 138 of cholangioscope 104 can have diameter D4 and can be configured to be as large as feasibly possible taking into account the amount of space required for optical components and other surgical components of cholangioscope 104. As such, lumen 138 can be configured to have one or more lumens 142 for other desired functionality of cholangioscope 104. Lumen 142 can be configured to receive components discussed with reference to FIGS. 5A and 5B, such as light transmitter 84, wiring 88 and fluid lines 89. Likewise, lumen 138 can be configured to accommodate pull wires 146A and 146B, which can be coupled to knob 38 (FIG. 3) in elongate body 120. In examples, D4 can be in the range of approximately 5.0 mm to approximately 6.0 mm. In additional examples, D4 can be in the range of approximately 2.0 mm to approximately 3.0 mm.

FIG. 3 is a schematic diagram of endoscopy system 10 comprising imaging and control system 12 and endoscope 14. The system of FIG. 3 is an illustrative example of an endoscopy system suitable for use with the systems, devices and methods described herein, such as direct peroral cholangioscope systems that can be used for obtaining samples of tissue or other biological matter to be removed from a patient for analysis or treatment of the patient. According to some examples, endoscope 14 can comprise cholangioscope 104 of FIGS. 1A-2 and can be insertable into an anatomical region for imaging and/or to provide passage of one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises an end-viewing cholangioscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by pull wires 146A and 146B connected to control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as tissue retrieval device 106 of FIGS. 1A and 1B. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as ports 40. Knob 38 can be coupled to pull wires 146A and 146B, or other actuation mechanisms, extending through insertion section 28 so that rotation of knob 38 can cause bending of functional section 30. Ports 40 can be configured to couple various electrical cables, guide wires, auxiliary scopes, tissue collection devices of the present disclosure, fluid tubes and the like to handle section 32 for coupling with insertion section 28. For example, cholangioscope 104 can be fed into endoscope 14 via one of ports 40.

Imaging and control system 12, according to examples, can be provided on a mobile platform (e.g., cart 41) with shelves for housing light source unit 22, suction pump 26, image processing unit 42 (FIG. 4), etc. Alternatively, several components of imaging and control system 12 shown in FIGS. 3 and 4 can be provided directly on endoscope 14 so as to make the endoscope “self-contained.”

Functional section 30 can comprise components for treating and diagnosing anatomy of a patient. Functional section 30 can comprise an imaging device, an illumination device and an elevator, such as is described further with reference to FIGS. 5A and 5B. Functional section 30 can comprise imaging and illuminating components configured for end-viewing, e.g., viewing distally or axially beyond of functional section 30.

FIG. 4 is a schematic diagram of endoscopy system 10 of FIG. 3 comprising imaging and control system 12 and endoscope 14. FIG. 4 schematically illustrates components of imaging and control system 12 coupled to endoscope 14, which in the illustrated example comprises an end-viewing cholangioscope. Imaging and control system 12 can comprise control unit 16, which can include or be coupled to image processing unit 42, treatment generator 44 and drive unit 46, as well as light source unit 22, input unit 20 and output unit 18.

Image processing unit 42 and light source unit 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 5A and 5B illustrate an example of functional section 30 of cholangioscope 14 of FIG. 4. FIG. 5A illustrates an end view of functional section 30 and FIG. 5B illustrates a cross-sectional view of functional section 30 taken along section plane 5B-5B of FIG. 5A. FIGS. 5A and 5B each illustrate “end-viewing endoscope” (e.g., gastroscope, colonoscope, cholangioscope, etc.) camera module 70. In end-viewing endoscope camera module 70, illumination and imaging systems are positioned such that the viewing angle of the imaging system corresponds to a target anatomy located adjacent an end of endoscope 14 and in line with central longitudinal axis A2 of endoscope 14.

In the example of FIGS. 5A and 5B, end-viewing endoscope camera module 70 can comprise housing 72, therapy unit 74, fluid outlets 76, illumination lens 78 and objective lens 80. Housing 72 can comprise and endcap for insertion section 28, thereby providing a seal to lumen 82.

As can be seen in FIG. 5B, insertion section 28 can comprise lumen 82 through which various components can be extended to connect functional section 30 with handle section 32 (FIG. 4). For example, illumination lens 78 can be connected to light transmitter 84, which can comprise a fiber optic cable or cable bundle extending to light source unit 22 (FIG. 4). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 4). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 4) and treatment generator 44 (FIG. 4). For example, therapy unit 74 can comprise a wide-diameter lumen for receiving other treatment components, such as cutting devices and therapeutic devices including tissue retrieval device 106.

Endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 4) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

FIG. 6 is a diagram illustrating guide sheath 102 and cholangioscope 104 of FIGS. 1A and 1B inserted perorally into patient 200 to reach duodenum 202. Guide sheath 102 can extend into mouth 204, through esophagus 206, through stomach 208 to reach duodenum 202. Before reaching intestines 210, guide sheath 102 can position cholangioscope 104 proximate common bile duct 212. Cholangioscope 104 can extend from guide sheath 102 to extend into common bile duct 212. Steering features of guide sheath 102, e.g., pull wires 40A and 40B can be used to facilitate navigating and bending of cholangioscope 104 through stomach 208, in addition to direct steering of cholangioscope 104 via pull wires 146A and 146B. For example, navigation of the Pyloric canal and Pyloric sphincter can be difficult to navigate using only an endoscope. Thus, guide sheath 102 can be used to turn or bend elongate body 120 of cholangioscope 104, or reduce the amount of steering or bending of elongate body 120 required by pull wires 146A and 146B, to facilitate traversing the Pyloric sphincter. Furthermore, once navigated through the Pyloric sphincter, another turn in the opposite direction is typically in order to enter duodenum 202. Again, the steering capabilities of guide sheath 102, which can be relaxed after navigating the pyloric sphincter, can again be employed to turn or bend elongate body 120 of cholangioscope 104 to reduce the burden on the native steering capabilities of cholangioscope 104. In view of the enhanced steering provided by guide sheath 102, the native steering capabilities of cholangioscope 104 (e.g., pull wires 146A and 146B) can be preserved for steering of elongate body 120 to direct tissue retrieval device 106 within duodenum 106.

FIG. 7 is a schematic illustration of a distal portion of guide sheath 102 having cholangioscope 104 and tissue collection device 106 extending therefrom according to the present disclosure. Guide sheath 102 can be positioned in duodenum 202, such as is described with reference to FIG. 6.

Duodenum 202 can comprise duct wall 214, sphincter of Oddi 216, common bile duct 212 and main pancreatic duct 218. Duodenum 202 comprises an upper part of the small intestine. Common bile duct 212 carries bile from the gallbladder and liver (not illustrated) and empties the bile into the duodenum 202 through sphincter of Oddi 216 via passage 220. Main pancreatic duct 218 carries pancreatic juice from the exocrine pancreas (not illustrated) to common bile duct 212. Sometimes it can be desirable to remove biological matter, e.g., tissue, from common bile duct 212 or pancreatic duct 218 to analyze the tissue to, for example, diagnose diseases or maladies of the patient such as cancer.

Cholangioscope 104 can be guided using guide sheath 102 such that a distal end of cholangioscope 104 is positioned proximate sphincter of Oddi 216. Therefrom, a surgeon can operate direct peroral cholangioscopy system 100 to extend tissue retrieval device 106 from cholangioscope 104 to enter common bile duct 212. The native steering capabilities of cholangioscope 104 can be used to turn cholangioscope 104, which in cases can be approximately ninety degrees, to face sphincter of Oddi 216. In examples, cholangioscope 104 can be advanced into common bile duct 212. In either case, tissue retrieval device 106 can be advanced form cholangioscope 104 and manipulated, such as by articulation of cholangioscope 104 using native steering capabilities, to gather target tissue within common bile duct 212. In particular, cholangioscope 104 can be used to navigate tissue retrieval device 106 toward the gall bladder, liver or other locations in the gastrointestinal system to perform various procedures. The surgeon can navigate tissue retrieval device 106 past entry 222 of main pancreatic duct 218 and into passage 220 of common bile duct 212, or into entry 222. Imaging capabilities of cholangioscope (e.g., a camera) can be used to view tissue retrieval device 106 to facilitate direct engagement with the target tissue.

In examples, lumen 138 of cholangioscope 104 can be used to deliver other devices to duodenum 202 and common bile duct 212 instead of or in addition to tissue retrieval device 106. The other devices can have their own functional capabilities, such as a light source, accessories, and biopsy channel, for therapeutic procedures.

Biological matter collected with tissue retrieval device 106 can be removed from the patient, typically by removal of tissue retrieval device 106 from cholangioscope 104, so that the removed biological matter can be analyzed to diagnose one or more conditions of the patient. According to several examples, tissue retrieval device 106 can be suitable for the removal of cancerous or pre-cancerous matter (e.g., carcinoma, sarcoma, myeloma, leukemia, lymphoma and the like), endometriosis evaluation, biliary ductal biopsies, and the like.

As discussed herein, the size of a typical tissue retrieval device is limited by the size of the auxiliary scope, which is itself limited by the size of a duodenoscope. As such, a typical tissue retrieval device can be on the order of approximately 1.2 mm or less. However, with the devices of the present disclosure, guide sheath 102 can be configured as a simple device such that the thickness of guide sheath can be small so that the working channel, e.g., lumen 136 can be large compared to that of a duodenoscope, which must additionally provide passageway for imaging, illuminating and fluid capabilities. As such, with conventional devices, it can be difficult to obtain sufficiently large tissue sample sized to ensure accurate diagnoses without having to repeatedly remove and reinsert the additional device. However, with the systems and devices of the present disclosure it is possible to obtain sufficiently large tissue sample sizes with only a single insertion and removal of the additional device (e.g., tissue retrieval device 106) due to, for example, lumen 138 being enlarged due to the small size of guide sheath 102.

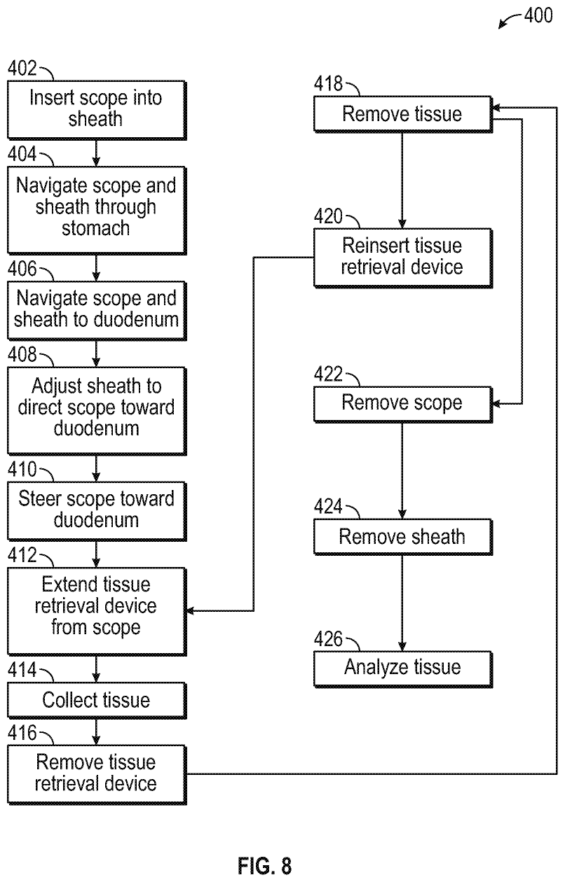

FIG. 8 is a block diagram illustrating examples of method 400 of collecting biological matter from a patient using direct peroral cholangioscopy system 100 of the present disclosure. Method 400 can encompass the use of guide sheath 102, cholangioscope 104 and tissue retrieval device 106 of FIGS. 1A and 1B.

At step 402, cholangioscope 104 can be inserted into guide sheath 102. Specifically, elongate body 120 of cholangioscope 104 can be inserted into lumen 136 of guide sheath 102. Cholangioscope 104 can be configured to freely slide within guide sheath 102 such that one can move relative to the other during insertion and thereafter.

At step 404, cholangioscope 104 and guide sheath 102 can be navigated through anatomy of a patient. Specifically, cholangioscope 104 and guide sheath 102 can be inserted into mouth 204 of the patient, pushed downward through esophagus 206 to stomach 208. Cholangioscope 104 and guide sheath 102 can be steered to extend through stomach 208 and into duodenum 202.

At step 406, cholangioscope 104 and guide sheath 102 can be pushed into duodenum 202. For example, guide sheath 102 can be steered, such as by using control knob 114 to pull at least one of pull wires 140A and 140B, to bend cholangioscope 104 to exit stomach 208 through the pyloric canal, thereby relieving controls of cholangioscope 104, e.g., pull wires 146A and 146B, from having to apply tension and/or compression to elongate body 120, further allowing cholangioscope 104 to have steerability capabilities for further use.

At step 408, guide sheath 102 can be adjusted, such as by using control knob 114 to pull at least one of pull wires 140A and 140B, to orient a distal end of guide sheath 102 toward Sphincter of Oddi 216, which again preserves native steering capabilities of cholangioscope 104, e.g., pull wires 146A and 146B, for later use. However, pull wires 146A and 146B of cholangioscope 104 can supplement action of pull wires 140A and 140B.

At step 410, cholangioscope 104 can be steered to duodenum 212 using pull wires 140A and 140B. Rigidity of guide sheath 102 can be used to allow cholangioscope 104 to push of guide sheath 102 to achieve the desired geometry to face towards and enter, if needed, duodenum 212. Cholangioscope 104 can be extended from guide sheath 102 to engage Sphincter of Oddi 216. In other examples, cholangioscope 104 can be extended from guide sheath 102 to penetrate Sphincter of Oddi 216.

At step 412, tissue retrieval device 106 can be inserted into cholangioscope 104. Tissue separator 128 can be extended from the distal end of elongate body 120 to engage tissue beyond Sphincter of Oddi 216 within common bile duct 212. In other examples, tissue retrieval device 106 can be guided through stomach 208 and duodenum 202 along with guide sheath 102 and cholangioscope 104.

At step 414, target tissue can be collected using tissue retrieval device 106. The target tissue can comprise tissue that is potentially diseased or otherwise indicative of a diseased condition of the patient. For example, separators 134 can be manipulated from control device 130 to engage target tissue one or more times to collect, separate if necessary, and store target tissue. In examples, tissue retrieval device 106 can be sized large enough, due to the factors discussed herein, to collect a sufficient volume of biological matter in a single collection operation such that multiple insertions of tissue retrieval device 106 can be avoided.

At step 416, tissue retrieval device 106 can be removed from within lumen 138 of cholangioscope 104. Tissue retrieval device 106 can be withdrawn so that target tissue can be removed form tissue separator 128.

At step 418, the target tissue can be removed from tissue separator 128. For example, separators 134 can be opened to allow access to the separated target tissue.

Thereafter, method 400 can return to step 412 via reinserting tissue retrieval device 106 into cholangioscope 104 at step 420 to collect additional matter from the previous target tissue site or collect additional matter from a different target tissue site, or can continue to step 422 to complete the operation.

At step 422, cholangioscope 104 can be removed from the patient, such as by withdrawal from guide sheath 102.

At step 424, guide sheath 102 can be removed from the patient, such as by withdrawal from esophagus 206.

At step 426, tissue collected form the patient can be analyzed, such as by being transported to a laboratory to be analyzed for the presence of diseased tissue, such as cancerous tissue.

As such, method 400 illustrates examples of methods of collecting biological matter from internal passages of a patient in large enough quantities for performing analysis without multiple passes, such as by using a tissue retrieval device having a size enabled by the use of a low-profile insertion guide sheath that does not include full functionality as a duodenoscope, such as imaging, illumination and fluid delivery capabilities. The low-profile insertion guide sheath also enables the size of a cholangioscope to be increased for acceptance of a large tissue retrieval device, and allows for the functionality of the cholangioscope, e.g., imaging, illumination and fluid delivery, to be leveraged throughout the insertion procedure (e.g., in the stomach), not just at or in the common bile duct.

### Various Notes & Examples

Example 1 is a direct peroral cholangioscope system comprising: a guide sheath comprising: a proximal end portion; a distal end portion; and a lumen extending between the proximal end portion and the distal end portion; wherein the guide sheath is sized to pass through an esophagus of a patient; an endoscope comprising: an elongate body extending from a proximal end to a distal end; an imaging device coupled to the elongate body proximate the distal end; and a passage extending at least partially through the elongate body and exiting at the distal end; wherein the elongate body is sized to pass through the lumen; and a tissue retrieval device configured to pass through the passage and retrieve tissue from within the patient.

In Example 2, the subject matter of Example 1 optionally includes the guide sheath further comprising steering capabilities.

In Example 3, the subject matter of Example 2 optionally includes the steering capabilities comprising a pull wire extending along the guide sheath.

In Example 4, the subject matter of Example 3 optionally includes the guide sheath comprising a distal bending section that comprises of a more flexible material than a proximal section.

In Example 5, the subject matter of Example 4 optionally includes the proximal section having a greater bending stiffness than the endoscope.

In Example 6, the subject matter of any one or more of Examples 2-5 optionally include the endoscope comprising native steering capabilities.

In Example 7, the subject matter of any one or more of Examples 1-6 optionally include the tissue retrieval device comprising forceps.

In Example 8, the subject matter of any one or more of Examples 1-7 optionally include an outer diameter of the guide sheath that is in the range of approximately 10 mm to approximately 12 mm.

In Example 9, the subject matter of Example 8 optionally includes a diameter of the elongate body that is in the range of approximately 8 mm to approximately 10 mm.

In Example 10, the subject matter of Example 9 optionally includes an outer diameter of the passage that is in the range of approximately 5 mm to approximately 6 mm.

Example 11 is a method of performing a direct peroral cholangioscopy procedure, the method comprising: inserting an endoscope into a guide sheath; inserting the guide sheath and endoscope into the stomach of a patient; inserting the guide sheath and endoscope into the duodenum of the patient; turning a distal end of the endoscope with the guide sheath to position the endoscope proximate the sphincter of Oddi; inserting a tissue retrieval device into the endoscope; positioning the tissue retrieval device in the common bile duct of the patient; and collecting biological matter with the tissue retrieval device.

In Example 12, the subject matter of Example 11 optionally includes turning a distal end of the endoscope with the guide sheath to position the endoscope proximate the sphincter of Oddi by tensioning a pull wire of the guide sheath.

In Example 13, the subject matter of Example 12 optionally includes tensioning a pull wire of the guide sheath by bending a distal bending section of the guide sheath with the pull wire.

In Example 14, the subject matter of any one or more of Examples 11-13 optionally include inserting the guide sheath and the endoscope into the duodenum of the patient by turning the endoscope with the guide sheath.

In Example 15, the subject matter of Example 14 optionally includes turning the endoscope with the guide sheath by tensioning a pull wire of the guide sheath.

In Example 16, the subject matter of any one or more of Examples 11-15 optionally include positioning the tissue retrieval device in the common bile duct of the patient by steering the endoscope with native steering capabilities.

In Example 17, the subject matter of Example 16 optionally includes inserting the endoscope into the sphincter of Oddi.

In Example 18, the subject matter of Example 17 optionally includes inserting the endoscope into the sphincter of Oddi by pushing the endoscope against the guide sheath.

In Example 19, the subject matter of any one or more of Examples 11-18 optionally include collecting biological matter with the tissue retrieval device by withdrawing the tissue retrieval device form the endoscope.

In Example 20, the subject matter of Example 19 optionally includes collecting biological matter with the tissue retrieval device by reinserting the tissue retrieval device to collect additional tissue.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A direct peroral cholangioscope system comprising:
a guide sheath comprising:
a proximal end portion;
a distal end portion; and
a lumen extending between the proximal end portion and the distal end portion;
wherein the guide sheath is sized to pass through an esophagus of a patient;
an endoscope comprising:
an elongate body extending from a proximal end to a distal end;
an imaging device coupled to the elongate body proximate the distal end; and
a passage extending at least partially through the elongate body and exiting at the distal end;
wherein the elongate body is sized to pass through the lumen; and
a tissue retrieval device configured to pass through the passage and retrieve tissue from within the patient.

2. The direct peroral cholangioscope system of claim 1, wherein the guide sheath further comprises steering capabilities.

3. The direct peroral cholangioscope system of claim 2, wherein the steering capabilities comprise a pull wire extending along the guide sheath.

4. The direct peroral cholangioscope system of claim 3, wherein the guide sheath comprises a distal bending section that comprises of a more flexible material than a proximal section.

5. The direct peroral cholangioscope system of claim 4, wherein the proximal section has a greater bending stiffness than the endoscope.

6. The direct peroral cholangioscope system of claim 2, wherein the endoscope comprises native steering capabilities.

7. The direct peroral cholangioscope system of claim 1, wherein the tissue retrieval device comprises forceps.

8. The direct peroral cholangioscope system of claim 1, wherein an outer diameter of the guide sheath is in the range of approximately 10 mm to approximately 12 mm.

9. The direct peroral cholangioscope system of claim 8, wherein a diameter of the elongate body is in the range of approximately 8 mm to approximately 10 mm.

10. The direct peroral cholangioscope system of claim 9, wherein an outer diameter of the passage is in the range of approximately 5 mm to approximately 6 mm.

11. A method of performing a direct peroral cholangioscopy procedure, the method comprising:
inserting an endoscope into a guide sheath;
inserting the guide sheath and endoscope into the stomach of a patient;
inserting the guide sheath and endoscope into the duodenum of the patient;
turning a distal end of the endoscope with the guide sheath to position the endoscope proximate the sphincter of Oddi;
inserting a tissue retrieval device into the endoscope;
positioning the tissue retrieval device in the common bile duct of the patient; and
collecting biological matter with the tissue retrieval device.

12. The method of claim 11, wherein turning a distal end of the endoscope with the guide sheath to position the endoscope proximate the sphincter of Oddi comprises tensioning a pull wire of the guide sheath.

13. The method of claim 12, wherein tensioning a pull wire of the guide sheath comprises bending a distal bending section of the guide sheath with the pull wire.

14. The method of claim 11, wherein inserting the guide sheath and the endoscope into the duodenum of the patient comprises turning the endoscope with the guide sheath.

15. The method of claim 14, wherein turning the endoscope with the guide sheath comprises tensioning a pull wire of the guide sheath.

16. The method of claim 11, wherein positioning the tissue retrieval device in the common bile duct of the patient comprises steering the endoscope with native steering capabilities.

17. The method of claim 16, further comprising inserting the endoscope into the sphincter of Oddi.

18. The method of claim 17, wherein inserting the endoscope into the sphincter of Oddi comprises pushing the endoscope against the guide sheath.

19. The method of claim 11, wherein collecting biological matter with the tissue retrieval device comprises withdrawing the tissue retrieval device form the endoscope.

20. The method of claim 19, wherein collecting biological matter with the tissue retrieval device further comprises reinserting the tissue retrieval device.
